(19) United States
(12) Patent Application Publication
(10) Pub. No.: US 2001/0046520 A1
(43) Pub. Date: Nov. 29, 2001

(54) Topical virucidal formulation

(75) Inventor: Joshua Berkowitz, Stanmore (GB)

Correspondence Address: BEYER WEAVER & THOMAS LLP, P.O. BOX 778, BERKELEY, CA, 94704-0778, US

(73) Assignee: Norton Healthcare Ltd.

(21) Appl. No.: 09/811,145

(22) Filed: Mar. 15, 2001

Related U.S. Application Data
(60) Provisional application No. 60/190,606, filed on Mar. 20, 2000.
(60) Provisional application No. 60/193,947, filed on Mar. 31, 2000.

(30) Foreign Application Priority Data
Mar. 15, 2000 (GB) 0006243
Mar. 28, 2000 (GB) 0007327.0

(51) Int. Cl. A61K 33/40; A61K 33/14

(52) U.S. Cl. 424/613; 424/680

(57) ABSTRACT

A topical virucidal pharmaceutical formulation comprising as active ingredient an inorganic halide, an oxidizing agent capable of generating hypohalite ions in aqueous solution, a sulphamic acid, a non-reducing organic acid and an inorganic phosphate optionally with a surfactant, preferably an anionic surfactant, together with one or more pharmaceutically acceptable excipients.

This invention relates to a topical virucidal formulation, particularly but not exclusively to a cream or other solid formulation for eradication or treatment of viral infections particularly on the skin or mucous membranes. The formulation may be used for therapeutic or prophylactic purposes. The invention also relates to ointments, pastes, emulsions, gels and rigid foam formulations.

A virucidal disinfectant composition for use with surgical equipment is disclosed in GB-A-2164851. This comprises and oxidising agent, for example potassium persulphate, sodium chloride, sulphamic acid, a non-reducing organic acid, an acid pyrophosphate and an anionic surfactant. Such disinfectants are sold under the trade mark VIRKON. There is no disclosure that this composition may be used in a pharmaceutical formulation.

According to a first aspect of the present invention a topical virucidal pharmaceutical formulation comprises as active ingredients an inorganic halide and an oxidising agent capable of generating hypohalite ions in aqueous solution, a sulphamic acid, a non-reducing organic acid and an inorganic phosphate optionally with a surfactant, preferably an anionic surfactant, together with one or more pharmaceutically acceptable excipients.

According to a second aspect of the present invention there is provided use of a composition for manufacture of a topical virucidal formulation, the composition comprising as ingredients an inorganic halide and an oxidising agent capable of generating hypohalite ions in aqueous solution, a sulphamic acid, a non-reducing organic acid and an inorganic phosphate optionally with a surfactant, preferably an anionic surfactant, together with one or more pharmaceutically acceptable excipients.

The oxidising agent is preferably a persulphate or pyroxiphthalate, particularly potassium persulphate. The inorganic halide is preferably sodium chloride.

The non-reducing organic acid may be selected from: malic acid, succinic acid and mixtures thereof.

The phosphate may be selected from disodium hydrogen pyrophosphate or potassium dihydrogen phosphate. Alternative anhydrous phosphates may be employed.

Any compatible pharmaceutically acceptable surfactant may be employed, for example sodium dodecylbenzene sulphonate, lauryl ethyl sulphonate and polyethylene oxide/polypropylene oxide condensates.

A preferred formulation available under the trade mark VIRKON comprises

| | |
|---|---|
| potassium persulphate (triple salt) | 50% |
| sodium chloride | 15% |
| sulphamic acid | 5% |
| malic acid | 10% |
| disodium pyrophosphate | 5% |
| anionic surfactant | 15% |
| | 100% |

The amount of the ingredients containing the active moeities is preferably a minimum amount necessary to provide virucidal efficacy. An amount of 0.1% to 40%, preferably 0.2% to 5%, preferably 1 to 3% by weight may be employed. Amounts and proportions in this specification are by weight unless otherwise indicated otherwise.

The formulation may comprise a cream wherein particles of the active-containing ingredients are uniformly dispersed. In order to ensure homogeneity of the product the particles may have a dimension less than 75 μm to provide a smooth cream having good dermal contact.

The formulation may include a hydrocarbon base, for example petrolatum or white ointment. An absorption base may be added, for example lanolin, lanolin isolates, cholesterol, lanosterol, acylated sterols or partial esters of polyhydric alcohols for example sorbitan monostearate or monooleate, or mixtures thereof.

Water removable bases are preferred to facilitate removal of the cream from the skin or clothing.

Creams in accordance with the present invention may incorporate one or more emulsifiers optionally together with one or more co-emulsifiers for example cetyl alcohol or glyceryl monostearate. Polyvalent ions, for example magnesium, calcium or aluminium may be added to stabilize the emulsion. A mixed emulsifier system maybe employed.

A humectant, for example glycerin, propyleneglycol or sorbitol may be employed to prevent drying of the cream on storage.

Formulations of the present invention find particular application as a prophylactic treatment for HIV or hepatitis infections for example as a vaginal cream or as a lining for surgical, dental, other medical or veterinary gloves.

An advantage arising from use of the present formulation is that the formulation is pink in colour. Loss of the pink colour before or during use indicates a loss of activity.

The invention is further described by means of example but not in any limitative sense.

### EXAMPLE 1

A formulation was prepared using the following ingredients:

| | |
|---|---|
| Aqueous cream BP | 495 g |
| Virkon | 5 g |
| | 500 g |

The resultant formulation was a pink cream.

### EXAMPLE 2

A formulation was prepared using:

| | | |
|---|---|---|
| Aqueous cream BP | 97.5 | g |
| Virkon | 2.5 | g |
| | 100 | g |

The resultant formulation was a pink cream. Tests were carried out to determine virucidal activity. Hepatitis A virus was used on account of its known resistance to virucides. The cream showed excellent efficacy.

What I claims is:

1. A topical virucidal pharmaceutical formulation comprising as active ingredient an inorganic halide, an oxidising agent capable of generating hypohalite ions in aqueous solution, a sulphamic acid, a non-reducing organic acid and an inorganic phosphate optionally with a surfactant, preferably an anionic surfactant, together with one or more pharmaceutically acceptable excipients.

2. A formulation as claimed in claim 1, wherein the oxidising agent is selected from the group consisting of persulphate and pyroxiphthalate.

3. A formulation as claimed in claim 2, wherein the oxidising agent is potassium persulphate.

4. A formulation as claimed in claim 1, wherein the non-reducing organic acid is selected from the group consisting of: malic acid, succinic acid and mixtures thereof.

5. A formulation as claimed in claim 1, wherein the phosphate is selected from the group consisting of disodium hydrogen phosphate and potassium dihydrogen phosphate.

6. A formulation as claimed in claim 1, wherein the surfactant is selected from the group consisting of sodium dodecylbenzene sulphonate, lauryl ethyl sulphonate and polyethylene oxide/polypropylene oxide condensates.

7. A formulation as claimed in claim 1, wherein the active ingredient consists of:

| | |
|---|---|
| potassium persulphate (triple salt) | 50% |
| sodium chloride | 15% |
| sulphamic acid | 5% |
| malic acid | 10% |
| disodium pyrophosphate | 5% |
| anionic surfactant | 15% |
| | 100% |

8. A formulation as claimed in claim 1, comprising 0.1 to 40% of active ingredient.

9. A formulation as claimed in claim 1, comprising 0.2 to 5% of active ingredient.

10. A formulation as claimed in claim 1, comprising 1 to 3% of active ingredient.

12. A formulation as claimed in claim 1, comprising a cream wherein particles of active ingredient have a dimension less than 75 μm.

13. A formulation as claimed in claim 1, comprising a cream including a hydrocarbon base.

14. A formulation as claimed in claim 13, further comprising an absorption base selected from the group consisting of lanolin, lanolin isolates, cholesterol, lanosterol, acylated sterols and partial esters of polyhydric alcohols.

15. A formulation as claimed in claim 13, comprising a cream incorporating one or more emulsifiers optionally together with one or more co-emulsifiers.

16. A hand cream comprising a formulation as claimed in claim 13.

17. A coating for an internal surface of a surgical, dental, veterinary or other medical glove comprising a formulation as claimed in claim 1.

18. A vaginal cream comprising a formulation as claimed in claim 1.

19. A surgical glove having a coating of a formulation as claimed in claim 1 on an internal surface thereof.

20. Use of a composition for manufacture of a topical virucidal formulation, the composition comprising a formulation as claimed in claim 1.
